(19) United States
(12) Patent Application Publication
(10) Pub. No.: US 2017/0035664 A1
(43) Pub. Date: Feb. 9, 2017

(54) FLAVOR-OPTIMIZED IONIZED WATER COMPOSITIONS

(71) Applicants: Mark E. Doherty, Taunton, MA (US); Russell R. Warwick, JR., East Freetown, MA (US); Jonathan Gordon, Narragansett, RI (US); Lea Rubinstein, Narragansett, RI (US)

(72) Inventors: Mark E. Doherty, Taunton, MA (US); Russell R. Warwick, JR., East Freetown, MA (US); Jonathan Gordon, Narragansett, RI (US); Lea Rubinstein, Narragansett, RI (US)

(21) Appl. No.: 15/224,581

(22) Filed: Jul. 31, 2016

Related U.S. Application Data
(60) Provisional application No. 62/200,630, filed on Aug. 3, 2015.

(51) Int. Cl. A61K 8/19; A61K 8/97; A61K 8/21; A23L 2/52; A61K 8/365; A61K 8/34; A23L 2/56; A23L 2/60; A61Q 11/00; A61K 8/25

(52) U.S. Cl. CPC A61K 8/19; A61Q 11/00; A61K 8/97; A61K 8/21; A61K 8/25; A61K 8/365; A61K 8/345; A23L 2/56; A23L 2/60; A23L 2/52; A23V 2002/00

(57) ABSTRACT

The present invention is directed to flavored ionized water compositions designed with effective concentrations of hydronium characterized by pH levels selected to improve oral health. In particular, the compositions of the present invention control the window of the effective concentration of hydronium in a flavored ionized water composition to afford novel flavored alkaline water compositions.

### RELATED APPLICATIONS

This application claims priority to U.S. Provisional Patent Application No. 62/200,630, filed on Aug. 3, 2015, under Attorney Docket No. RMX-001-1; the entirety of which is incorporated herein by reference.

### BACKGROUND OF THE INVENTION

A clear trend of increased sugar consumption and poor oral health habits has emerged throughout the global population, and very often leads to oral infection, pain, low self-esteem, malnutrition, speech difficulties, absenteeism at work and school, and poor overall health. In fact, years of evidence based scientific studies have confirmed the direct links of poor oral health, e.g., cavities or periodontal disease, to diabetes, heart disease, hypertension, stroke, inflammation, lung disease, kidney disease, pre-term births, prosthetic joint complications, Alzheimer's, and obesity. Moreover, these studies demonstrate the overall importance of maintaining healthy mouths and good nutrition. The evidence that proves the relationship between increased consumption of sugar sweetened beverages and chronic disease is so overwhelming that some states have considered or actually added extra taxes to these products in an effort to deter people from consuming these beverages.

The average American now consumes an alarming 50 gallons per person per year of sugar sweetened beverages. This increase has occurred equally across every age, sex and race. While poor oral health leads to poor overall health in all populations, it is especially acute for populations marked by low-socio economic status. For example, in the year 2000, the Surgeon General's Report: Oral Health in America reported that a “silent epidemic of oral diseases is affecting our most vulnerable citizens—poor children, the elderly, and many members of racial and ethnic minority groups.” Moreover, the prevalence of obesity has been rising steadily over the last several decades and is currently at unprecedented levels: more than 68% of US adults are considered overweight, and 35% are obese, and almost 7% are morbidly obese.

More than the link to overall health, dental caries is the most common chronic infectious disease in children and adolescents (i.e., with respect to diseases caused by bacteria and transmittable from person to person). It is 5 times more common than asthma, and 7 times more common than hay fever. In addition, children of low-income families and racial and ethnic minorities suffer twice as much dental caries when compared to more affluent socio-economic populations. Moreover, this trend has been shown to be coincident with drinking sugar sweetened beverages one or more times per day.

As a consequence to these globally disturbing concerns, there is a current prevailing movement at both the government and personal levels to promote wellness and maintenance of healthy eating habits. In this way, the consumer has looked to replace their consumption of soda and other related soft drinks with water compositions, including flavored and functional water (i.e., water containing additives that provide extra nutritional value), as well as juice products. Such products include, for example, Vitamin Water®, Gatorade® and even reduced calorie juices such as Snapple® and Nantucket Nectars®, which still retain staggering amounts of sugar and unwanted calories. As a step further, given that the American Heart Association recommends no more than 6 teaspoons (30 grams) of sugar a day for women and 9 teaspoons (45 grams) for men, artificially sweetened beverages with significantly reduced calories have begun to replace these products. However, current scientific understanding reveals that even artificially sweetened with sugar-substitutes, the present functional water market offers products that do not necessarily promote healthy outcomes; in fact the evidence shows that they actually contribute to the diseases previously mentioned as related to poor oral health.

Certain specialized ionized water compositions have recently emerged on the market that are considered to offer advantages over traditional functional water. Along with a growing list of advantages, for athletes, it has been considered to hydrate the body quicker compared to other beverages. Such advantages have also been shown to include protection against neural cellular oxidative damage, as well as general oxidative damage to DNA, RNA, and protein; suppressive effect on apoptosis; diabetes mellitus; type I diabetes; type II diabetes; amelioration of inflammation; inhibition of acute ethanol-induced hangovers; and even an enhanced apoptosis-inducing effect on certain cancer cells.

Moreover, the utilization of alkaline ionized water compositions (i.e., water compositions with pH greater than 7) has been argued to neutralize acid in your bloodstream, boost your metabolism, as well as help your body absorb nutrients more effectively; and studies have actually shown that it can act as an adjunct treatment of the acidity in disorders such as acid reflux. Even though opponents of this argument suggest that the body is self-balancing without such alkaline water, products of this nature have remained essentially unmodified, and therefore less appealing to consumers, for the fundamental reason that achieving the greater alkalinity levels becomes more difficult with the addition of functional components, and especially flavors that would improve the taste of the ionized water compositions. Such additives unavoidably and significantly reduce the pH levels of the alkaline ionized water.

As such, there exists a need for alternative alkaline water compositions that take advantage of the benefits of ionized water.

### SUMMARY OF THE INVENTION

Accordingly, the present invention is directed to ionized water compositions designed with effective concentrations of hydronium with pH levels selected to improve oral health. In particular, the compositions of the present invention control the window of the effective concentration of hydronium in a flavored ionized water composition to afford novel flavored alkaline water compositions. The flavored alkaline water compositions are characterized by pH levels defined as greater than one and a half times those of the putative “critical pH” of the Stephan curve (shown in FIG. 1) at which point it has been postulated enamel dissolves, and below which acidic bacteria thrive.

The present invention has identified an effective concentration window (ECW) of hydronium for the improvement of oral health, and the treatment of oral disease. Moreover, the present invention, in order to selectively design these flavored compositions to promote oral health, has identified a method to flavor such compositions without increasing the effective concentration of hydronium that would result in compositions with a pH measurement below the “super-critical pH” level, or the lower end of the ECW described herein. For example, the ECW of hydronium in these compositions characterized by a pH of 8.5 to 10 may be achieved through the utilization of an optimized flavoring agent. Given that the improvement of oral health is well known to improve overall health, the compositions of the present invention which afford increased oral health, also reduce oral disease, and in turn, improve the overall health of the consumer.

Accordingly, one aspect of the invention is a flavor-optimized ionized water composition. The composition comprises alkaline ionized water selected to achieve a controlled effective concentration window (ECW) of hydronium in the composition characterized by a pH of 8.5 to 10, e.g., 9.1 to 9.5; and an optimized flavoring agent suitable to maintain the hydronium concentration within the ECW after combination with the alkaline ionized water.

Another aspect of the invention provides a flavor-optimized ionized water composition comprising alkaline ionized water selected to achieve a controlled effective concentration window (ECW) of hydronium in the composition characterized by a pH of 8.5-10, e.g., 9.1 to 9.5; an optimized flavoring agent suitable to maintain the hydronium concentration within the ECW after combination with the alkaline ionized water; and additional components suitable to maintain the hydronium concentration within the ECW comprising xylitol, Fluorosilicic Acid, Calcium Lactate Gluconate, and Monk Fruit Extract.

In an additional aspect, the present invention provides a method of using a composition of the present invention for improving the health of an individual.

In yet another aspect, the present invention provides a method of using a composition of the present invention for improving the dental health of an individual.

In yet another aspect, the present invention provides a method of using a composition of the present invention in combination with a pH measuring tool for improving the dental health of an individual.

### BRIEF DESCRIPTION OF THE DRAWINGS

Advantages of the present apparatus will be apparent from the following detailed description, which description should be considered in combination with the accompanying drawings, which are not intended limit the scope of the invention in any way.

FIG. 1 depicts the well-known Stephan Curve, which shows the effects of a sucrose rinse on the pH level in the oral cavity. As depicted, the pH level quickly drops below the critical pH minutes after the rinse; highlighting the importance of avoidance of, or recovery from, this response before the amount of time available for a net loss of tooth structure becomes too great.

### DETAILED DESCRIPTION OF THE INVENTION

Although most people understand that sugar in the beverages we drink contributes to poor oral health, it is not as well known that the acidity in such beverages also has a substantial impact on oral health; and in many cases, a far more significant one. In fact, beverages with acidic pH create an acidic environment in the mouth allowing “bad bacteria” in the mouth to flourish and overpopulate while reducing “good bacteria” (i.e., beneficial bacteria, which normally provide a physically protective layer on our teeth) increasing vulnerability of the teeth to erosion, thus resulting in oral disease, such as dental cavities (caries) and gum disease. Moreover, once oral pH drops below 5.5, commonly referred to as the “critical point,” the cavity (caries) process ensues.

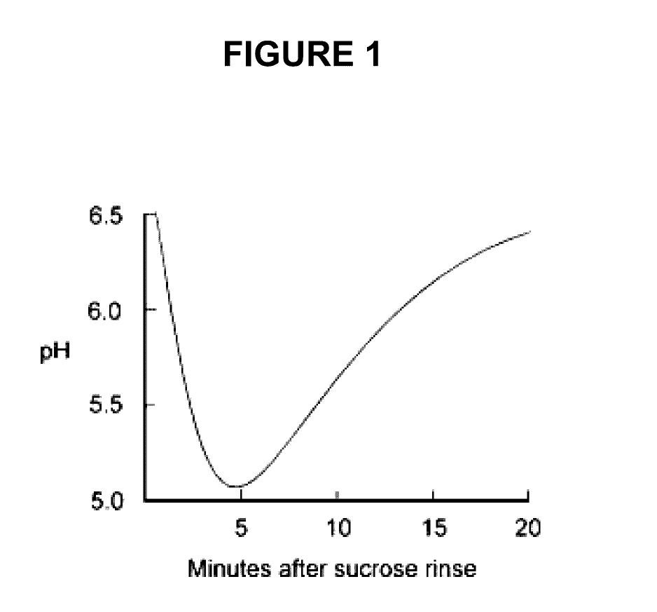

The present invention not only recognizes the importance of eliminating sugar based sweeteners, but also recognizes the substantial impact of the pH level of the beverage. Accordingly, the present invention is directed to ionized water compositions designed with effective concentrations of hydronium with pH levels selected to improve oral health. In particular, the compositions of the present invention control the window of the effective concentration of hydronium in a flavored ionized water composition to afford novel flavored alkaline water compositions. The flavored alkaline water compositions are characterized by basic pH levels; with the pH levels, in certain embodiments, defined as greater than one and a half times those of the putative “critical pH” of the Stephan curve (shown in FIG. 1) at which point it has been postulated enamel dissolves, and below which acidic bacteria thrive. This is in stark contrast to the current market of beverages that possess strongly acidic pH levels of 2, 3, and 4.

The present invention, including compositions and related methods will be described with reference to the following definitions that, for convenience, are set forth below. Unless otherwise specified, the below terms used herein are defined as follows:

### I. Definitions

As used herein, the term “a,” “an,” “the” and similar terms used in the context of the present invention (especially in the context of the claims) are to be construed to cover both the singular and plural unless otherwise indicated herein or clearly contradicted by the context.

The terms “acid” and “acidity” are art-recognized, and describes a substance or a characteristic of a substance that donates hydrogen ions, or in water, where the hydronium ions exceed the hydroxide ions.

The terms “base” and “alkaline” are art-recognized, and describe a substance or a characteristic of a substance that accepts hydrogen ions, or in water, where the hydroxide ions exceed the hydronium ions. The relative alkaline or base nature of the substance may be described in terms of its “alkalinity” or “basicity,” which are also art-recognized terms.

The term “concentration” is art-recognized as the amount of a substance in a unit amount of another substance or defined space; and, for example, may be expressed in terms of moles/L or molarity. In certain embodiments, the concentration of hydronium may be described as its logarithmic relationship with pH: where pH is the negative logarithm of the hydrogen ion (i.e., hydronium ion) concentration:

pH=−log [H⁺]

(where the square brackets around the H+ indicate the “concentration” expressed in molarity). As such, for each single unit change in pH (i.e., a change by 1), the hydrogen ion concentration changes ten-fold, wherein the pH scale runs from 0 to 14. pH values lower than 7 are acidic, pH values higher than 7 are alkaline; and pure water has a neutral pH of 7. In certain embodiments of the present invention, the hydronium concentration is characterized by a pH of 8.5 to 10.

The term “controlled” as used herein, for example in the language “controlled effective concentration window (ECW)” of hydronium, is used to describe the action of the selection of materials to achieve an identified (i.e., pre-determined) result where, for example, maintaining the composition within the ECW by active selection would be characterized as controlling the effective concentration window. In certain embodiments of the invention, the components are selected specifically in order to achieve a concentration of hydronium characterized by a pH of 8.5 to 10. This act of selection, may be considered an additional step in the methods of certain embodiments of the invention.

The language “flavoring agent” is art-recognized, and is used herein to describe substances added to foods (e.g., drinks) to improve the quality of taste.

The term “effective” as used in the expression “effective concentration window” is used herein to describe the concentration window that produces a successful or desired result, e.g., the improvement in oral health, described herein.

The term “hydronium” is an art-recognized ionically charged molecular species formed when a hydrogen ion (H+) combines with a water molecule (H₂O) to form H₃O⁺. It is a well-known equivalent representation of a hydrogen ion in aqueous acidic solution. In fact, for simplicity, hydronium, or hydronium ions, are often referred to as hydrogen ions H⁺. In pure water, there are an equal number of hydronium ions and hydroxide ions. However, in alkaline water, the hydronium ions present are less than the hydroxide ions present (which means, for example, that increasing the hydronium concentration in a composition would reduce the pH).

The term “optimized,” as used in the expression “optimized flavoring agent” is used herein to describe the flavoring agents of the present invention selected to be suitable to maintain the hydronium concentration within the ECW after combination with the alkaline ionized water. For example, in certain embodiments such selection as optimized may be made based on the type of agent used. In certain embodiments, the selection as optimized may be related to the amount of agent used. In certain embodiments, the selection as optimized may be based on type and amount used.

The language “oral health” and “dental health” are used interchangeably herein to describe the health of the oral cavity with respect to disease or disorders caused or affected by bacteria of the oral cavity, for example, periodontal disease or cavities.

The language “oral cavity” is art-recognized, and describes the cavity of the mouth, bounded by the jaw bones and associated structures (muscles and mucosa).

The term “suitable” is used herein to describe the components of the composition selected to maintain the hydronium concentration within the ECW after combination with the alkaline ionized water. In certain embodiments, a component that is not, by itself considered suitable, may be combined with another component/agent to afford an aggregate set of components that is suitable to maintain the hydronium concentration within the ECW after combination with the alkaline ionized water.

The term “window” as used herein, is art-recognized to describe a range with a top and a bottom, inclusive. In the certain embodiments of the present invention, the pH window is defined on its lower end by the “super-critical pH” level, and establishes the maximum effective concentration of hydronium.

II. Ionized Water Compositions of the Invention

One embodiment of the present invention provides a flavor-optimized ionized water composition comprising
alkaline ionized water selected to achieve a controlled effective concentration window (ECW) of hydronium in the composition characterized by a pH of 8.5 to 10, e.g., 9 to 10, e.g., 9.1 to 9.5; and
an optimized flavoring agent suitable to maintain the hydronium concentration within the ECW after combination with the alkaline ionized water. In certain embodiments, the water is ionized through the process of electrolysis. The ionized water is selected to achieve a controlled effective concentration window (ECW) of hydronium in the composition, e.g., characterized by a pH of 8.5 to 10. In the mouth, this alkaline environment is selected to reduce and/or eliminate the growth of the “bad bacteria” that cause dental disease, e.g., neutralizing the acidity in the oral cavity, aiding in the prevention of oral disease.

In the mouth, the changes over time in response to a challenge (usually a cariogenic food) may be graphically represented by the Stephan curve also known as Stephan responses. The pH of dental plaque under resting conditions (i.e., when no food or drink has been consumed), is fairly constant (although differences do exist, however, between individuals and in different sites within an individual). The Stephan curve highlights a critical pH level of 5.5, below which point it has been postulated enamel dissolves, and below which acidic bacteria thrive. The response after exposure of dental plaque to a fermentable carbohydrate is that pH decreases rapidly (for example, below the critical pH level) reaching a minimum in approximately 5 to 20 minutes. This is followed by a gradual recovery to its starting value, usually over 30 to 60 minutes (although this can be longer in some individuals).

In contrast, the present invention addresses the challenges posed by acidogenic bacteria in dental plaque that rapidly metabolize fermentable carbohydrates producing acidic end products. The flavored alkaline water compositions are characterized by pH levels defined herein equal to or greater than the “super-critical pH” level, which are pH levels greater than one and a half times those of the putative “critical pH” of the Stephan curve.

The basic or alkaline oral environment created by the consumption of the compositions of the present invention favors re-mineralization of teeth undergoing the early cavity process (i.e., rebuilding of tooth structure), rather than de-mineralization (i.e., cavities: breakdown of tooth structure) which may be caused by drinks high in sugar and acidity.

In another embodiment, the present invention provides a flavor-optimized ionized water composition comprising
alkaline ionized water selected to achieve a controlled effective concentration window (ECW) of hydronium in the composition characterized by a pH of 8.5 to 10, e.g., 9 to 10, e.g., 9.1 to 9.5;
an optimized flavoring agent suitable to maintain the hydronium concentration within the ECW after combination with the alkaline ionized water; and
additional components selected to achieve the controlled ECW of hydronium in the composition comprising xylitol, Fluorosilicic Acid, Calcium Lactate Gluconate, and Monk Fruit Extract.

A. Ionized water

The ionized water useful in making the compositions of the present invention is alkaline ionized water. In certain embodiments, the ionized water is obtained through the process of electrolysis, e.g., electrodialysis. In particular embodiments, the electrolysis is performed using a water ionizer, e.g., a water ionizer designed to reach alkaline pH of 10 or greater. In certain embodiments, the invention provides a large scale modified water ionizer modified to reach alkaline pH of 10 or greater; in contrast to the large scale water ionizers currently available that produce a maximum alkaline pH of 9.5, which on their own (unmodified to achieve the desired pH of 10 or greater) may not be suitable for the preparation of the compositions of the present invention on a large scale. As such, in an additional embodiment, the present invention provides a water ionizer designed to achieve alkaline ionized water of pH 10 or greater, e.g., in large scale production.

In certain embodiments, a water ionizer may be used to separate water into alkaline and acid fractions using a process known as electrolysis. The electrolysis process makes both alkaline and acidic waters at the same time, which are then separated. Moreover, the electrochemical process in which water is split to form hydrogen and oxygen by an electric current may be shown chemically as:

2 H₂O(/)→2 H₂(g)+O₂(g)

When the above process occurs in pure water, hydronium (or more simply, H⁺) cations will accumulate at the anode and OH⁻ anions will accumulate at the cathode. As such, during this process the water near the anode is acidic while the water near the cathode is alkaline. Drawing the water from near the cathode affords alkaline ionized water useful for the compositions of the present invention.

In certain embodiments, an electrolyte e.g., calcium ions, may be added prior to electrolysis, for example, to reduce the energy needed for electrolysis.

B. Effective Concentration Window

The present invention has identified an effective concentration window (ECW) of hydronium for the improvement of oral health, and the treatment of oral disease. Moreover, the present invention, in order to selectively design these flavored compositions to promote oral health, has identified a method to flavor such compositions without increasing the effective concentration of hydronium that would result in compositions with a pH measurement below the “super-critical pH” level, or the lower end of the ECW described herein. For example, the ECW of hydronium in these flavored compositions characterized by a pH of 8.5 to 10 may be achieved through the utilization of an optimized flavoring agent.

Given that the improvement of oral health is well known to improve overall health, the compositions of the present invention which afford increased oral health, also reduce oral disease, and in turn, improve the overall health of the consumer.

In certain embodiments of the present invention, the controlled effective concentration window (ECW) of hydronium is characterized by a pH of 9 to 10.

In certain embodiments of the present invention, the controlled effective concentration window (ECW) of hydronium characterized by a pH of 9.1 to 9.5.

C. Optimized Flavoring Agent

The flavoring agents of the present invention are optimized in order to ensure the ECW of hydronium is maintained when combined with the remainder of the components in the composition. In certain embodiments, a flavoring agent that is not, by itself considered optimized, may be combined with another agent, or further modified to afford an aggregate optimized flavoring agent that is suitable to maintain the hydronium concentration within the ECW after combination with the alkaline ionized water.

In certain embodiments of the present invention, the optimized flavoring agent is present in 0.1% A to 0.2% weight by weight.

In certain embodiments of the present invention, the optimized flavoring agent is Flavor, Coconut (SF-8619, Sovereign), e.g., 0.16%.

In certain embodiments of the present invention, the optimized flavoring agent is Flavor, Coconut Milk (35583, David Michael), e.g., 0.16%.

In certain embodiments of the present invention, the optimized flavoring agent is Flavor, Ripe Mango (FY2165, Ungerer), e.g., 0.15%.

In certain embodiments of the present invention, the optimized flavoring agent is Flavor Cantaloupe (FY2173, Ungerer), e.g., 0.10%.

In certain embodiments of the present invention, the optimized flavoring agent is Flavor, Watermelon (FY2169, Ungerer), e.g., 0.10%.

In certain embodiments of the present invention, the optimized flavoring agent is Flavor, Grape (FY5150, Ungerer), e.g., 0.2%.

D. Sweeteners

In certain embodiments of the present invention, the additional component in the composition of the present invention is one or more sweetening agents.

In certain embodiments, the sweetening agent is Monk Fruit Extract.

In certain embodiments, the sweetening agent is selected from one or more of the non-nutritive sweeteners acesulfame-K, aspartame, neotame, saccharin, and sucralose, formulated in an amount and in a manner suitable to produce a final composition with a pH within the effective concentration windows of hydronium described herein.

In certain embodiments, the sweetening agent may also act as a functional component. In particular embodiments, the sweetening agent is xylitol. Xylitol is a naturally occurring five-carbon sugar polyol, and is a Food and Drug Administration (FDA) approved sweetener, and has 33% fewer calories than sugar. It has been extensively studied over the last 40 years for its effect on dental caries. It is naturally found in vegetables, fruit, and berries, and may be artificially manufactured from xylan-rich plant materials, such as birch and beechwood. Studies have shown that xylitol breaks up plaque in the mouth and stimulates salivary flow allowing for re-mineralization of tooth structure and reduction in bacterial load. In particular, it is believed that Xylitol reduces the levels of mutans streptococci (MS) in plaque and saliva by disrupting their energy production processes, leading to wasted energy cycle and cell death. As such, xylitol has been shown to reduce plaque (which contributes to cavities) and helps stop cavities from forming and progressing. It also reduces the adhesion of MS to the teeth surface as well as reduces their acid production potential.

Moreover, the benefits of xylitol do not stop in the oral cavity. Xylitol alcohol has also been shown to impact growth of nasopharyngeal bacteria such as S. pneumonia and S. mitis, and hence has a role to play in nasopharyngeal pneumonia. While xylitol has no antibacterial properties of its own; it appears to enhance the body's own innate immunity. In fact, xylitol has anti-adhesive effects on micro-organisms like Streptococcus pneumoniae and Streptococcus mutans, inhibiting their growth. It has further been shown to reduce the incidence of ear infections in children and increase bone volume/density, especially when combined with calcium.

Although at high dosages, xylitol can cause diarrhea in children at 45 g/day and 100 g/day in adults, the amount tolerated varies with individual susceptibility and body weight. Most adults can tolerate 40 g/day, but the recommended dose for dental caries prevention is 6-10 g/day. As such, in certain embodiments, the amount of xylitol is less than 40 g/day. In certain embodiments, the amount of xylitol is equal to or less than 6-10 g/day.

Given the use of xylitol for these many know indications, the compositions presented herein that comprise xylitol will also be useful for such indications.

E. Additional Functional Components

The compositions of the present invention may further include additional functional components that add advantages to the compositions.

In certain embodiments of the present invention, the compositions further comprise one or more additional components suitable to maintain the hydronium concentration within the ECW, e.g., after combination with the alkaline ionized water. In particular embodiments, the components suitable for maintaining the hydronium concentration within the ECW are used for long-term stabilization of the pH corresponding to the ECW, e.g., during storage (e.g., for the shelf-life of the composition). In certain embodiments, the additional component may be selected from minerals and electrolytes, for example, selected from the group consisting of sodium bicarbonate, dipotassium phosphate, calcium chloride, magnesium sulfate, or any combination thereof. In particular embodiments, the additional component may be selected from minerals and electrolytes, for example, selected from the group consisting of dipotassium phosphate, calcium chloride, magnesium sulfate, or any combination thereof.

In certain embodiments, the additional component is one or more dental health components. In particular embodiments, the dental health component is selected from the group consisting of xylitol, a fluoride contributing component, a calcium contributing component, and any combination thereof. In certain embodiments, the amount of fluoride is 0.3 ppm, (e.g., which is less than the amount in a typical glass of fluoridated tap water). Fluoride helps prevent tooth decay by making the tooth structure harder. Studies also show that it inhibits growth of bacteria. Calcium supports the structure and formation of bones and teeth.

In certain embodiments, wherein further comprising xylitol, a fluoride contributing component, and a calcium contributing component.

In certain embodiments, wherein the fluoride contributing component is Fluorosilicic Acid.

In certain embodiments, wherein the calcium contributing component is Calcium Lactate Gluconate.

III. Methods of Use of the Compositions of the Invention

Another embodiment of the present invention provides a method of using a composition of the present invention for improving the dental health of an individual.

In an additional embodiment, the present invention provides a method of using a composition of the present invention for improving the health of an individual.

In certain embodiments, such health improvements comprise those overall health advantages associated with the improvement of oral health.

In certain embodiments, such health improvements comprise those overall health advantages associated with the components of the composition. For example, the health improvements may also include those advantages associated with ionized water or xylitol. Such health advantages include, but are not limited to one or more of the following: reduction of plaque; reduction of formation and progression of cavities; reduction of oral bacteria acid production; treatment of the acidity of disorders such as acid reflux; protection against neural cellular oxidative damage, as well as general oxidative damage to DNA, RNA, and protein; suppressive effect on apoptosis; diabetes mellitus; type I diabetes; type II diabetes; amelioration of inflammation; and enhanced apoptosis-inducing effect on certain cancer cells.

Another embodiment of the present invention provides a method of using a composition of the present invention in combination with a pH measuring tool for improving the dental health of an individual. Such pH measuring tool is any device that may be suitable for measuring pH (e.g., pH strips, testing solution, or a device). Moreover, the pH measuring tool may be used to guide the timing and/or amount of a composition of the present invention that a consumer should consume.

IV. Methods of Preparation of the Compositions of the Invention

The compositions of the present invention are not intended to be limited by the methods used in the preparation of the compositions. However, in certain embodiments, the present invention provides novel methods to achieve the large scale production of the flavor-optimized ionized water compositions of the present invention. Such large scale production may be facilitated by the use of large scale water ionizers designed to achieve alkaline ionized water at a pH of 10 or greater.

### Exemplification

Having thus described the invention in general terms, reference will now be made to the accompanying drawings of exemplary embodiments, which are not necessarily drawn to scale, and which are not intended to be limiting in any way.

In this respect, it is to be understood that the invention is not limited in its application to the details of production or the specific compositions/components set forth in the following description. The invention is capable of other embodiments and of being practiced and carried out in various ways. Also, it is to be understood that the phraseology and terminology employed herein are for the purpose of description and should not be regarded as limiting.

### EXAMPLE 1

### Flavor-Optimized Ionized Water Compositions

The ionized water was prepared as alkaline ionized water, e.g., from electrodialysis of pure water, and combined with the xylitol, fluorosilicic acid, calcium lactate gluconate, and monk fruit extract in the percentages noted in Table 1 in a weight by weight fashion; wherein the ionized water was used to make up the 100% composition (i.e., further account for the optimized flavoring agent added, and not shown in Table 1).

TABLE 1

| | Percentage of |
|---|---|
| Component | Composition |
| Ionized Water | 97.3382422% |
| Xylitol | 2.0000000% |
| Fluorosilicic Acid (19%), Mosaic | 0.0001578% |
| Calcium Lactate Gluconate | 0.6400000% |
| Monk Fruit Extract | 0.0216000% |
| | 100.0000000% |

### Ionized Alkaline Water

An electronic water ionizer was used to create alkaline water; such system can be used on an industrial scale as well. The system separated the water into two streams and the alkaline water is collected.

The ionizing unit operates by diverting water into the alkaline water system, where it first passes through an activated carbon filter to help remove tap water pollutants (chlorine, fluoride, benzenes, pesticides, etc.). Finally, the water passes over platinum coated titanium plates that have a positive and negative charge—created by a “matching” power supply. By doing this electrolysis the positive and negative charge separates the water, where more oxygen, i.e., negative OH ions, get attracted to the positive charge (Alkaline); and more hydrogen gets attracted to the negative charge HO (Acidic).

Optimized Flavoring Agent

Examples of optimized flavoring agents suitable to maintain the hydronium concentration within the ECW after combination with the alkaline ionized water are reported in Table 2. Each optimized flavoring agent was added to the combination of components described in Table 1; wherein the ionized water was used to make up the 100% composition. The final pH of the composition comprising the optimized flavoring agent is also shown in Table 2 as maintained within the ECW.

TABLE 2

| Flavors (Source) | Final Composition pH |
|---|---|
| Flavor, Coconut (SF-8619., | 9.32 |
| Sovereign) @ 0.16% | |
| Flavor, Coconut Milk (35583, David | 9.35 |
| Michael) @ 0.16% | |
| Flavor, Ripe Mango (FY2165, | 9.24 |
| Ungerer) @ 0.15% | |
| Flavor Cantaloupe (FY2173, Ungerer) | 9.41 |
| @ 0.10% | |
| Flavor, Watermelon (FY2169, | 9.35 |
| Ungerer) @ 0.10% | |
| Flavor, Grape (FY5150, Ungerer) @ | 9.27 |
| 0.2% | |

### EXAMPLE 2

### Evaluation and Selection of Components Suitable to Achieve Effective Concentration Window of Hydronium

Evaluation and selection of components was performed based on the desire to control and maintain the hydronium concentration within the effective concentration window (ECW) after combination. Selecting the pH of the materials based, and controlling the pH of the combination products, required a pre-selected recognition of the oral health benefits. Such selection involved the proper initial election of pH of the alkaline ionized water; consideration of the effect of additional components, particularly flavoring agents; and desired functional advantages for the final composition.

Additional experiments provided a greater understanding of the ability to achieve certain functional goals for the final composition. Evaluation of phosphorus, calcium, vitamin D, fluoride, xylitol, and the pH of the alkaline ionized water was performed as described below.

### Evaluation of Phosphorus:

Phosphorus was evaluated for inclusion within the compositions of the present invention. The following sources of phosphate were examined by combination with alkaline ionized water and calcium:

Phosphorus Source
Monopotassium phosphate
Nutrifos
polyphosphates:
dipotassium phosphate
Sodium Potassium Hexametaphosphate

Based on these experiments, it was concluded the combination of phosphorus and calcium was not stable in the current exemplary compositions, which resulted in the creation of phosphoric acid, precipitation, or unacceptable pH levels.

### Evaluation of Calcium:

Calcium lactate gluconate was determined in the studies to be a very soluble and bioavailable form of calcium

### Evaluation of pH of Alkaline Ionized Water:

It was determined that to achieve a final composition pH of the current exemplary compositions within the ECW, and greater than 9, the starting pH of the ionized water was in the range of about pH 10 to 10.24, e.g., about 10.14 to 10.24. In certain embodiments, the alkaline ionized water was achieved using an Aqua Ionizer Deluxe, e.g., 7.0.

### Evaluation of Fluoride:

Fluoride levels were verified as safe per FDA CFR 165.110(b)(4)(ii), and were confirmed as acceptable in the form of Fluorosilicic Acid (19%), Mosaic.

### Evaluation of Xylitol:

Xylitol levels were determined based on well tolerated levels from a gastrointestinal point of view. For example, 2% weight by weight was elected.

### Evaluation of Vitamin D:

It was determined that vitamin D was not soluble in the current exemplary compositions.

### Evaluation of Flavors:

Evaluation of over 64 different flavors was performed based on pH profiles to achieve conformation with the desired effective concentration window of hydronium in the final composition. Six (6) different flavors profiles of the 64 were identified as suitable.

### REFERENCES

1.) Oral health Quality Improvement in the age of accountability. December 2011.A Report Prepared by: Paul Glassman DDS, MA, MBA Professor of Dental Practice, Director of Community Oral Health, and Director of the Pacific Center for Special Care at University of the Pacific Arthur A. Dugoni School of Dentistry.

2.) Sugar Sweetened Beverages, Obesity, Type 2 Diabetes and

Cardiovascular Disease risk. Circulation. 2010 Mar. 23; 121(11): 1356-1364. doi: 10.1161/CIRCULATIONAHA.109.876185. Vasanti S. Malik, MSc, Barry M. Popkin, PhD, George A. Bray, MD, Jean-Pierre Després, PhD, and Frank B. Hu, MD, PhD Circulation. 2010 Mar. 23; 121(11): 1356-1364.

3.) Sugar-sweetened beverages and risk of obesity and type 2 diabetes: Epidemiologic evidence

Physiol Behay. 2010 Apr. 26; 100(1): 47-54. Published online 2010 Feb. 6. doi: 10.1016/j.physbeh.2010.01.036Frank B. Hu, MD, PhD1,2 and Vasanti S. Malik, MSc1

4.) The Public Health and Economic Benefits of Taxing Sugar-Sweetened Beverages. N Engl J Med. Author manuscript; available in PMC 2011 Jul. 20. Published in final edited form as: N Engl J Med. 2009 Oct. 15; 361(16): 1599-1605. Published online 2009 Sep. 16. doi: 10.1056/NEJMhpr0905723Dr. Kelly D. Brownell, Ph.D., Professor and director, Dr. Thomas Farley, M.D., M.P.H., Health commissioner, Dr. Walter C. Willett, M.D., Dr.P.H., Professor and chair, Dr. Barry M. Popkin, Ph.D., Professor, Dr. Frank J. Chaloupka, Ph.D., Professor of economics, Dr. Joseph W. Thompson, M.D., M.P.H., Associate professor, and Dr. David S. Ludwig, M.D., Ph.D., Director

5.) Periodontal disease and systemic conditions: a bidirectional relationship. Odontology. Author manuscript; available in PMC 2008 Jul. 7.Published in final edited form as: Odontology. 2006 September; 94(1): 10-21. doi: 10.1007/s10266-006-0060-6. Jemin Kim and Salomon Amar

6.) Effect of Periodontal Treatment on Glycemic Control of Diabetic Patients. Diabetes Care. 2010 February; 33(2): 421-427. doi: 10.2337/dc09-1378. A systematic review and meta-analysis. Wijnand J. Teeuw, DDS,1 Victor E. A. Gerdes, PHD,2,3 and Bruno G. Loos, PHD1

7.) Impact of Poor Oral Health on Children's School Attendance and Performance. Am J Public Health. 2011 October; 101(10): 1900-1906. doi: 10.2105/AJPH.2010.200915. PMCID: PMC3222359. Stephanie L. Jackson, DDS, M S, William F. Vann, Jr, DMD, PhD, Jonathan B. Kotch, MD, MPH, Bhavna T. Pahel, PhD, MPH, BDS, and Jessica Y. Lee, DDS, PhD, MPH

8.) Oral Health: The Silent Epidemic. Public Health Rep. 2010 March-April; 125(2): 158-159. PMCID: PMC2821841. Regina M. Benjamin, MD, MBA

9.) The Impact of Oral Health on the Academic Performance of Disadvantaged Children. Am J Public Health. 2012 September; 102(9): 1729-1734. Published online 2012 September. doi: 10.2105/AJPH.2011.300478. PMCID: PMC3482021. Hazem Seirawan, DDS, MPH, MS, Sharon Faust, DDS, and Roseann Mulligan, DDS, MS. Br J Sports Med. 2013 November; 47(16):1054-8. doi: 10.1136/bjsports-2013-092891. Epub 2013 Sep. 2

10.) Oral health and impact on performance of athletes participating in the London 2012 Olympic Games: a cross-sectional study. Br J Sports Med doi:10.1136/bjsports-2013-092891 Needleman 11, Ashley P, Petrie A, Fortune F, Turner W, Jones J, Niggli J, Engebretsen L, Budgett R, Donos N, Clough T, Porter S.

11.) Poor oral health is associated with coronary heart disease and elevated systemic inflammatory and haemostatic factors. Clin Periodontol. 2004 January; 31(1):25-9. Montebugnoli L1, Servidio D, Miaton R A, Prati C, Tricoci P, Melloni C. J Acad Nutr Diet. Author manuscript; available in PMC 2014 Jan. 1.

12.) CONSUMPTION PATTERNS OF SUGAR SWEETENED BEVERAGES IN THE UNITED STATES. J Acad Nutr Diet. 2013 January; 113(1): 43-53. doi: 10.1016/j.jand.2012.09.016. Euna Han1 and Lisa M. Powell2

13.) Intake of sugar-sweetened beverages and weight gain: a systematic review1,2,3. Am J Clin Nutr. Author manuscript; available in PMC 2011 Nov. 8. Published in final edited form as: Am J Clin Nutr. 2006 August; 84(2): 274-288. Vasanti S Malik, Matthias B Schulze, and Frank B Hu. J Acad Nutr Diet. 2013 January; 113(1):43-53. doi: 10.1016/j.jand.2012.09.016. Obesity (Silver Spring). 2006 October; 14(10):1825-31.

14.) Self-reported sugar-sweetened beverage intake among college students. Obesity (Silver Spring). 2006 October; 14(10):1825-3i.West DS1, Bursac Z, Quimby D, Prewitt T E, Spatz T, Nash C, Mays G, Eddings K.

15.) Bacterial signatures in thrombus aspirates of patients with myocardial infarction. Indian Heart J. 2013 September; 65(5): 632-633. doi: 10.1016/j.ihj.2013.08.008. Reviewed by Suraj Khanal*Tanja Pessi, Vesa Karhunen, Pasi P. Karjalainen, Antti Ylitalo, Juhani K. Airaksinen, Matti Niemi, Mikko Pietila, Kari Lounatmaa, Teppo Haapaniemi, Terho Lehtimaki, Reijo Laaksonen, Pekka J. Karhunen, Jussi Mikkelsson Bacterial signatures in thrombus aspirates of patients with myocardial infarction Circulation Vol. 127; 2013 1219 1228

16.) Potential benefits of pH 8.8 alkaline drinking water as an adjunct in the treatment of reflux disease. Ann Otol Rhinol Laryngol. 2012 July; 121(7):431-4.Koufman JAI, Johnston N.

17.) Electrolyzed-reduced water protects against oxidative damage to DNA, RNA, and protein. Appl Biochem Biotechnol. 2006 November; 135(2):133-44. Lee MY1, Kim Y K, Ryoo K K, Lee Y B, Park E J.

18.) Anti-diabetic effects of electrolyzed reduced water in streptozotocin-induced and genetic diabetic mice. Life Sci. 2006 Nov. 10; 79(24):2288-92. Epub 2006 Aug. 2. Kim MJ1, Kim H K.

19.) Suppressive effects of electrolyzed reduced water on alloxan-induced apoptosis and type 1 diabetes mellitus. Cytotechnology. 2011 March; 63(2): 119-131. Published online 2010 Nov. 10. doi: 10.1007/s10616-010-9317-6. Yupin Li,1,2 Takeki Hamasaki,1 Noboru Nakamichi,3 Taichi Kashiwagi,1 Takaaki Komatsu,1 Jun Ye,1,4 Kiichiro Teruya,1,5 Masumi Abe,1 Hanxu Yan,5 Tomoya Kinjo,5 Shigeru Kabayama,3 Munenori Kawamura,6 and Sanetaka Shirahata 1,5

20.) Electrolyzed-reduced water scavenges active oxygen species and protects DNA from oxidative damage. Biochem Biophys Res Commun. 1997 May 8; 234(1):269-74. Shirahata Si, Kabayama S, Nakano M, Miura T, Kusumoto K, Gotoh M, Hayashi H, Otsubo K, Morisawa S, Katakura Y.

21.) Electrochemically Reduced Water Protects Neural Cells from Oxidative Damage. Oxid Med Cell Longev. 2014; 2014: 869121. Published online 2014 Oct. 14. doi: 10.1155/2014/869121. Taichi Kashiwagi, 1 Hanxu Yan, 2 Takeki Hamasaki, 2 Tomoya Kinjo, 2 Noboru Nakamichi, 1 Kiichiro Teruya, 1, 2 Shigeru Kabayama, 3 and Sanetaka Shirahata 1, 2, *

22.) Recent Progress Toward Hydrogen Medicine: Potential of

Molecular Hydrogen for Preventive and Therapeutic Applications. Curr Pharm Des. 2011 July; 17(22): 2241-2252. doi: 10.2174/138161211797052664. Shigeo Ohta*

23.) Acidic beverages increase the risk of in vitro tooth erosion. Nutr Res. Author manuscript; available in PMC 2009 Jan. 1. Published in final edited form as: Nutr Res. 2008; 28(5): 299-303. doi: 10.1016/j.nutres.2008.03.001. Leslie A. Ehlen, Teresa A. Marshall, 1 Fang Qian, James S. Wefel, and John J. Warren

24.) Acidic diet and dental erosion among athletes. Aust Dent J. 2002 September; 47(3):228-36. Sirimaharaj V1, Brearley Messer L, Morgan M V.

25.) Dental erosion and severe tooth decay related to soft drinks: a case report and literature review*. J Zhejiang Univ Sci B. 2009 May; 10(5): 395-399. doi: 10.1631/jzus.B0820245. Ran Cheng, Hui Yang, Mei-ying Shao, Tao Hu, †‡ and Xue-dong Zhou.

26.) Effects of Soft Drink Consumption on Nutrition and Health: A

Systematic Review and Meta-Analysis. Am J Public Health. 2007 April; 97(4): 667-675. doi: 10.2105/AJPH.2005.083782. Lenny R. Vartanian, PhD, Marlene B. Schwartz, PhD, and Kelly D. Brownell, PhD

27.) Dental caries and beverage consumption in young children. Pediatrics. 2003 September; 112(3 Pt 1):e184-91. Marshall TA1, Levy S M, Broffitt B, Warren J J, Eichenberger-Gilmore J M, Burns T L, Stumbo P J.

28.) The effect of pH on the erosion of dentine and enamel by dietary acids in vitro. J Oral Rehabil. 2001 September; 28(9):860-4. West NX1, Hughes J A, Addy M.

29.) Added sugar intake and cardiovascular diseases mortality among US adults. JAMA Intern Med. 2014 April; 174(4):516-24. doi: 10.1001/jamainternmed.2013.13563. Yang Q1, Zhang Z1, Gregg EW2, Flanders WD3, Merritt R1, Hu FB4.

30.) Dietary sugars intake and cardiovascular health: a scientific statement from the American Heart Association. Circulation. 2009 Sep. 15; 120(11):1011-20. doi: 10.1161/CIRCULATIONAHA.109.192627. Epub 2009 Aug. 24. Johnson R K, Appel L J, Brands M, Howard B V, Lefevre M, Lustig R H, Sacks F, Steffen L M, Wylie-Rosett J; American Heart Association Nutrition Committee of the Council on Nutrition, Physical Activity, and Metabolism and the Council on Epidemiology and Prevention

31.) Oxidative stress as a mechanism of added sugar-induced cardiovascular disease. Int J Angiol. 2014 December; 23(4):217-26. doi: 10.1055/s-0034-1387169. Prasad K1, Dhar 12.

32.) Sugar sweetened beverages consumption and risk of coronary heart disease: a meta-analysis of prospective studies. Atherosclerosis. 2014 May; 234(1):11-6. doi: 10.1016/j.atherosclerosis.2014.01.037. Epub 2014 Feb. 15.Huang C1, Huang J1, Tian Y1, Yang X1, Gu D2.

33.) Gastro-oesophageal reflux and dental erosion. Evid Based Dent. 2008; 9(2):54. doi: 10.1038/sj.ebd.6400586. Milosevic A1.

34.) The Surgeon General's report on America's oral health: opportunities for the dental profession. J Am Dent Assoc. 2000 December; 131(12):1721-8.Evans CA1, Kleinman D V.

35.) The U.S. Oral Health Workforce in the Coming Decade: Workshop Summary. Institute of Medicine (US) Board on Health Care Services. Washington (DC): National Academies Press (US); 2009.

36.) The Connection Between Oral Health and Overall Health and Well-Being. Caswell A. Evans, Jr., D.D.S., M.P.H.University of Illinois at Chicago, College of DentistryPediatr Clin North Am. 2000 October; 47(5):1149-57.

37.) Relationships between oral and systemic health. Pediatr Clin North Am. 2000 October; 47(5):1149-57.Casamassimo PS1.

38.) Periodontal Disease and Coronary Heart Disease Incidence: A Systematic Review and Meta-analysis. J Gen Intern Med. 2008 December; 23(12): 2079-2086. Published online 2008 Sep. 20. doi: 10.1007/s11606-008-0787-6. Linda L. Humphrey, MD, MPH, 1,2,3,4,7,8 Rongwei Fu, PhD,1,4,5 David I. Buckley, MD, MPH,1,2,6 Michele Freeman, MPH,1 and Mark Helfand, MD, MPH1,2,3,7

39.) Relationship between oral health and mortality in cardiovascular diseases. J Clin Periodontol. 2001 August; 28(8):762-8. [Article in English, French, German]Jansson L1, Laystedt S, Frithiof L, Theobald H.

40.) Oral health in women with coronary heart disease. J Periodontol. 2005 April; 76(4):544-50. Buhlin K1, Gustafsson A, Ahnve S, Janszky I, Tabrizi F, Klinge B.

41.) Periodontitis and diabetes: a two-way relationship. Diabetologia. 2012 January; 55(1): 21-31. Published online 2011 Nov. 6. doi: 10.1007/s00125-011-2342-y. P. M. Preshaw, 1 A. L. Alba,2 D. Herrera,3 S. Jepsen,4 A. Konstantinidis,5 K. Makrilakis,6 and R. Taylor7

42.) Periodontal disease and the oral-systemic connection: “is it all the RAGE?”. Quintessence Int. 2010 March; 41(3):229-37.Katz J1, Wallet S, Cha S.

43.) Global Bottled Water Market to Reach $195 Billion in 2018; Flavored/Functional Water to Show Greatest Growth Jul. 15, 2014. Additional Reports Wellesley, Mass., Jul. 15, 2014—BCC.

Research(www.bccresearch.com)

44.) Intake of sugar-sweetened beverages and weight gain: a systematic review1,2,3. American Journal of Clinical Nutrition. Vasanti S Malik, Matthias B Schulze, and Frank B Hu

45.) Consumption of Sugar-Sweetened Beverages Among US Adults in 6 States: Behavioral Risk Factor Surveillance System, 2011. Sohyun Park, PhD; Liping Pan, MD, MPH; Bettylou Sherry, PhD, RD; Heidi M. Blanck, PhDSuggested citation for this article: Park S, Pan L, Sherry B, Blanck H M. Consumption of Sugar-Sweetened Beverages Among US Adults in 6 States: Behavioral Risk Factor Surveillance System, 2011. Prey Chronic Dis 2014; 11:130304. DOI: http://dx.doi.org/10.5888/pcd11.130304 .

46.) http://www.cdc.gov/obesity/data/adult.html

47.) http://www.nyam.org/policy/take-action/sp-sbb.html New York Academy of Medicine

48.) Anti-diabetic effects of electrolyzed reduced water in streptozotocin-induced and genetic diabetic mice. Life Sci. 2006 Nov. 10; 79(24):2288-92. Epub 2006 Aug. 2.Kim MJ1, Kim H K.

49.) Ionized alkaline water: new strategy for management of metabolic acidosis in experimental animals. Ther Apher Dial. 2009 June; 13(3):220-4. doi: 10.1111/j.1744-9987.2009.00659.x.Abol-Enein H1, Gheith O A, Barakat N, Nour E, Sharaf A E.

50.) Dose-dependent inhibition of gastric injury by hydrogen in alkaline electrolyzed drinking water. BMC Complement Altern Med. 2014; 14: 81. Published online 2014 Mar. 3. doi: 10.1186/1472-6882-14-81Jinling Xue,1 Guodong Shang,1 Yoshinori Tanaka,3 Yasuhiro Saihara,3 Lingyan Hou,1 Natalia Velasquez,1 Wenjun Liu,1,2 and Yun Lu 1,2

51.) Drinking Hydrogen Water Ameliorated Cognitive Impairment in Senescence-Accelerated Mice. J Clin Biochem Nutr. 2010 May; 46(3): 269-276. Published online 2010 Apr. 23. doi: 10.3164/jcbn.10-19. Yeunhwa Gu,1 Chien-Sheng Huang,2,3 Tota Inoue,1 Takenori Yamashita,1 Torao Ishida,1 Ki-Mun Kang,4 and Atsunori Nakao3,*

52.) Hydrogen Gas Presents a Promising Therapeutic Strategy for Sepsis. Biomed Res Int. 2014; 2014: 807635. Published online 2014 Apr. 16. doi: 10.1155/2014/807635Keliang Xie, 1, 2 Lingling Liu, 1, 2 Yonghao Yu, 1, 2, * and Guolin Wang 1, 2

53.) Remineralization effects of xylitol on demineralized enamel. J Electron Microsc (Tokyo). 2003; 52(5):471-6.Miake Y1, Saeki Y, Takahashi M, Yanagisawa T.

54.) The effect of xylitol on dental caries and oral flora. Clin Cosmet Investig Dent. 2014; 6: 89-94. Published online 2014 Nov. 10. doi: 10.2147/CCIDE.S55761Prathibha Anand Nayak,1 Ullal Anand Nayak,2 and Vishal Khandelwal3

55.) Xylitol and dental caries: an overview for clinicians. J Calif Dent Assoc. 2003 March; 31(3):205-9. Lynch H1, Milgrom P.

56.) Xylitol chewing gum and dental caries. Int Dent J. 1995 February; 45(1 Suppl 1):65-76.Tanzer JM1.

57.) A novel use of xylitol sugar in preventing acute otitis media. Pediatrics. 1998 October; 102(4 Pt 1):879-84.Uhari M1, Kontiokari T, Niemela M.

58.) Increased bone volume and bone mineral content in xylitol-fed aged rats.

Gerontology. 2001 November-December; 47(6):300-5.Mattila PT1, Svanberg M J, Knuuttila M L.

59.) Dietary xylitol supplementation prevents osteoporotic changes in streptozotocin-diabetic rats.

Metabolism. 1998 May; 47(5):578-83.Mattila PT1, Knuuttila M L, Svanberg MJ.

60.) Antimicrobial actions of fluoride for oral bacteria. Can J Microbiol. 1995 November; 41(11):955-64.Marquis RE1.

61.) Biochemical effects of fluoride on oral bacteria.

J Dent Res. 1990 February; 69 Spec No:660-7; discussion 682-3.Hamilton IR1.

62.) Electrolyzed-reduced water inhibits acute ethanol-induced hangovers in Sprague-Dawley rats. Biomed Res. 2009 October; 30(5):263-9. Park SK1 , Qi X F, Song S B, Kim D H, Teng Y C, Yoon Y S, Kim K Y, Li J H, Jin D, Lee K J.

### Incorporation By Reference

The entire contents of all patents, published patent applications and other references cited herein are hereby expressly incorporated herein in their entireties by reference.

### Equivalents

Those skilled in the art will recognize, or be able to ascertain using no more than routine experimentation, numerous equivalents to the specific procedures described herein. Such equivalents were considered to be within the scope of this invention and are covered by the following claims. Moreover, any numerical or alphabetical ranges provided herein are intended to include both the upper and lower value of those ranges. In addition, any listing or grouping is intended, at least in one embodiment, to represent a shorthand or convenient manner of listing independent embodiments; as such, each member of the list should be considered a separate embodiment.

What is claimed is:

1. A flavor-optimized ionized water composition comprising
alkaline ionized water selected to achieve a controlled effective concentration window (ECW) of hydronium in the composition characterized by a pH of 8.5 to 10; and
an optimized flavoring agent suitable to maintain the hydronium concentration within the ECW after combination with the alkaline ionized water.

2. The composition of claim 1, wherein the controlled effective concentration window (ECW) of hydronium is characterized by a pH of 9 to 10.

3. The composition of claim 1, wherein the controlled effective concentration window (ECW) of hydronium characterized by a pH of 9.1 to 9.5.

4. The composition of claim 1, wherein the optimized flavoring agent is present in 0.1% to 0.2% weight by weight.

5. The composition of claim 1, further comprising one or more additional components suitable to maintain the hydronium concentration within the ECW.

6. The composition of claim 5, wherein the additional component is a sweetening agent.

7. The composition of claim 6, wherein the sweetening agent is Monk Fruit Extract.

8. The composition of claim 5, wherein the additional component is one or more dental health components.

9. The composition of claim 8, wherein the dental health component is selected from the group consisting of xylitol, a fluoride contributing component, a calcium contributing component, and any combination thereof.

10. The composition of claim 9, wherein the fluoride contributing component is Fluorosilicic Acid.

11. The composition of claim 9, wherein the calcium contributing component is Calcium Lactate Gluconate.

12. The composition of claim 1, further comprising xylitol, a fluoride contributing component, and a calcium contributing component.

13. A flavor-optimized ionized water composition comprising
alkaline ionized water selected to achieve a controlled effective concentration window (ECW) of hydronium in the composition characterized by a pH of 8.5 to 10;
an optimized flavoring agent suitable to maintain the hydronium concentration within the ECW after combination with the alkaline ionized water; and
additional components suitable to maintain the hydronium concentration within the ECW comprising xylitol, Fluorosilicic Acid, Calcium Lactate Gluconate, and Monk Fruit Extract.

14. The composition of claim 13, wherein the controlled effective concentration window (ECW) of hydronium characterized by a pH of 9.1 to 9.5.

15. The composition of claim 13, wherein the optimized flavoring agent is present in 0.1% to 0.2% weight by weight.

16. A method of using a composition of claim 1 for improving the health of an individual.

17. A method of using a composition of claim 1 for improving the dental health of an individual.

18. A method of using a composition of claim 1 in combination with a pH measuring tool for improving the dental health of an individual.
